(19) United States
(12) Patent Application Publication
(10) Pub. No.: US 2010/0008982 A1
(43) Pub. Date: Jan. 14, 2010

(54) NON-COVALENT COMPLEXES OF BIOACTIVE AGENTS WITH STARCH FOR ORAL DELIVERY

(76) Inventors: Eyal Shimoni, Haifa (IL); Uri Lesmes, Nazareth Ilit (IL); Yael Ungar, Atzmon (IL)

Correspondence Address: WINSTON & STRAWN LLP;PATENT DEPARTMENT, 1700 K STREET, N.W., WASHINGTON, DC, 20006, US

(21) Appl. No.: 12/298,162

(22) PCT Filed: Apr. 25, 2007
(86) PCT No.: PCT/IL2007/000511

§ 371 Date: Mar. 26, 2009

Related U.S. Application Data
(60) Provisional application No. 60/794,110, filed on Apr. 24, 2006.

(51) Int. Cl. A61K 9/48; A61K 9/14; A61K 9/20; A61K 31/20; A61P 35/00

(52) U.S. Cl. 424/451; 424/489; 424/464; 514/560

(57) ABSTRACT

The present invention relates to particles comprising non-covalent complexes which comprise starch and an active agent and dry compositions containing such particles for the oral delivery of the active agents. Preferably the particles are degraded and release the active agent within the intestines, protecting the active agent from degradation in the stomach. The present invention further relates to methods for preparing suspensions of these particles having a uniform particle size in the range of several microns.

### FIELD OF THE INVENTION

The present invention relates to particles comprising non-covalent complexes comprising starch and an active agent, methods for preparing same providing a uniform size in the nanoscale or microscale range, and dry compositions comprising the particles useful for oral delivery of the active agents.

### BACKGROUND OF THE INVENTION

The formulation of pharmaceuticals and nutraceuticals in compositions providing good oral bioavailability combined with stability of the active ingredient in a cost effective manner is a major pursuit of the pharmaceutical sciences. The choice of excipients and the determination of suitable particle sizes are among the most relevant considerations during development of any oral formulation containing particles, whether in liquid or dry form.

Among the many excipients that are approved for use in pharmaceuticals and nutraceuticals and/or generally recognized as safe, starches, including both natural and modified, are among the most widely used.

Numerous scientific studies have supported the concept that nutritional intervention is strongly associated with genetic expression patterns, which are responsible for a variety of biological functions. Based on these findings, the effects of dietary supplements and nutrients have been investigated enthusiastically mainly due to their cost effectiveness. One advocated approach is the supplementation of diets with nutraceuticals, which are natural, bioactive chemical compounds. However, their introduction into foods has proved to be a major technological challenge since many nutraceuticals have high sensitivity to light, heat and oxidation. Because of these shortcomings further applications of dietary supplements have stagnated. This is partially due to a lack of basic awareness to drug delivery systems and rational design thereof, especially with food grade biopolymers, as a means to deliver efficiently and control the release of nutraceuticals in the gastrointestinal tract.

Recently amylose-lipid complexes were used as controlled lipid release agents to protect the bioactive fatty acids (FAs) octadecanoic acid (also known as conjugated linoleic acid or CLA) and docosahexadecanoic acid (DHA) (Lalush et al. Biomacromolecules, 6: 121-130, 2005). These complexes were found to form spherical aggregates with diameter at the nanoscale (1-100 nm) and micrososcale (100-1000 nm) (Lalush et al., ibid.).

Amylose as a polysaccharide delivery system. Naturally occurring in plants, amylose is used as macro or micro delivery system to protect bioactive molecules from the hostile conditions of the upper gastrointestinal (GI) tract. Amylose breaks down in the lower GI, whether by human enzymes or saccharolytic bacteria that trigger the release of the encapsulated bioactive agents, thus enabling controlled and targeted delivery of the bioactive agents on site (Mehvar et al. Curr. Pharm. Biotechnol. 4: 283-302, 2003). Amylose is a food grade homopolymer of α(1-4) linked D-glucopyranose that tends to form a hollow helix form (termed V-amylose) known to host a variety of compounds, ranging from iodine atoms to large molecules such as fatty acids, phenols and mono- or di-glycerides (Tufvesson et al. Starch, 55: 138-148, 2003). This form has recently gained attention as a carrier of small organic molecules, aroma compounds, and bioactive agents (Le Bail et al. Int. J. Biol. Macromol. 35: 1-7, 2005; Kawada et al. Starch, 56: 13-19, 2004).

Molecular properties of V-amylose. V-amylose has a relatively large central cavity with a pitch of about 8 Å per turn and an adjustable diameter of 6, 7 or 8 glycosyl groups per turn depending on the size of the guest molecule. It is often assumed that the guest molecule is a “stem” inside the helix whose inner surface is hydrophobic because of the carbon-hydrogen matrix provided by the helically wound α (1-4) glucan. Theoretical modeling suggests that when V-amylose hosts fatty acids it forms an imperfect helix with the fatty acid partly inside, partly out, placing the carboxyl head outside the V-helix, leaving only the glycosidic C(4)-O—C(1) bonds as the greatest points of the helix flexibility. It has also been suggested that two main polymorph forms of V-complex exist, namely type I and type II (a and b). These polymorph forms are mainly characterized by the temperature of their dissociation determined by differential Scanning Calorimetry (DSC), and X-ray diffraction (XRD), both suggest that type I has lower crystallinity. Transmission electron microscopy (TEM) of complexes with saturated FAs of up to 16 carbons demonstrated uniaxial layout of amylose molecules locally interrupted by amorphous segments. Amylose degree of polymerization (DP), solution pH, complexation temperature and the structure of the complexed lipid (e.g., monoglyceride or FA) affect complex formation as well as its thermal stability, which increases with FA chain length and decreases with instauration, both in the case of monoglycerides and FA. Other factors such as concentration ratios, duration of complexation time, water content, concentration of amylose and that of the FA are also of importance. Various studies as well as Nuclear Magnetic Resonance (NMR) studies have aided in the development of a suggested mechanism of formation and a model structure of the complexes (FIG. 1).

Effects of chemical modifications. Influencing the functional properties as well as the digestibility of starches is possible through various chemical and physical modifications of starches. For example, cationization of the polysaccharide schizophyllan, a natural beta-(1-->3)-D-glucan, was shown to convert it to a useful carrier of antisense oligonucleotides exhibiting increased efficiency of cellular uptake and higher thermostability (Matsumoto et al. Biochim. Biophys. Acta, 1670: 91-104, 2001).

Under FDA regulations, cationized starch can only be used in paper or paperboards in contact with food while starches that undergo limited and controlled oxidation, esterification, cross linking or etherification are permitted for use as food additives (Cui, S. W. (2005) Food Carbohydrates: chemistry, physical properties, and applications. 3 edition. Boca Raton, Fla. Taylor and Francis). Studies have shown that such modifications of starch affect its supramolecular structure, functionality and digestibility.

Preparation of Amylose-Conjugated Linoleic Acid (CLA) Complexes in water/dimethylsulfoxide solution or in KOH/HCL solution was shown to yield complexes resistant against oxidation, which retain CLA in simulated stomach conditions (Lalush et al. ibid.). In the presence of pancreatin, these complexes were found to release CLA, suggesting that amylose-CLA complexes can serve as molecular nanocapsules for protection and delivery of CLA.

U.S. Pat. No. 4,911,952 teaches encapsulation methods of biological agents by entrapment the biological agents within matrix of unmodified starch. According to U.S. Pat. No. 4,911,952 the starch is prepared for encapsulation by dispersing it in water and passing the dispersion through a stream-injection cooker at a temperature of about 120°-135° C. so that essentially all the amylose molecules of the starch are dissociated. Preferred method for gelatinization is stream-injection cooking, although extrusion cooking is also taught.

U.S. Pat. No. 5,955,101 discloses starch as a complexant with iodine for preparing dry powder pharmaceutical formulations useful in the preparation of capsules or tablets. U.S. Pat. No. 5,955,101 further discloses a process for preparing a starch-iodine complex characterized by exposing starch to aqueous molecular iodine at 20° C. for sufficient period of time to allow complexation.

U.S. Pat. No. 5,910,318 discloses methods for treating an iodine deficiency disorder in a patient by orally administering a pill or a capsule comprising a therapeutically effective amount of a non-covalent starch-iodine complex to said patient, wherein the starch in the starch-iodine complex contains from 20% to 100% amylose.

U.S. Pat. No. 6,482,413 discloses complexes for oral delivery of drugs, therapeutic proteins and peptides, and vaccines. According to U.S. Pat. No. 6,482,413, Vitamin B₁₂ or its analogs are covalently coupled to micro or nano particles in which a bioactive agent is entrapped. According to U.S. Pat. No. 6,482,413 the micro or nano particles include polysaccharide polymers such as starch, pectin, amylose, guar gum, dextran, and other natural and semi synthetic derivatives of polysaccharides. U.S. Pat. No. 6,482,413 further discloses a method of modifying a micro or nano particle carrier for delivery of injectable drugs, therapeutic proteins and peptides in order to make it suitable for oral delivery, the method comprising coupling to said carrier a vitamin B₁₂ to form a complex U.S. Pat. No. 6,994,869 discloses enteral formulations for nasogastric delivery comprising an amino acid source, a carbohydrate source, a lipid source, and a fatty acid delivery agent, wherein the fatty acid delivery agent being a fatty acid covalently bonded to a carrier molecule by a bond hydrolysable in the colon, said carrier being a starch, a non-starch polysaccharide, or oligosaccharide. U.S. Pat. No. 6,994,869 further discloses a method for elevating the level of a fatty acid in the colon comprising a step of delivering a fatty acid delivery agent in a physiologically acceptable medium through a feeding tube.

U.S. Pat. No. 6,878,693 discloses hydrophilic inclusion complexes consisting essentially of nano-sized particles of a water-insoluble lipophilic compound surrounded by or entrapped within an amphiphilic polymer. U.S. Pat. No. 6,878,693 further discloses a method for forming the hydrophilic inclusion complexes comprising adding a low concentration solution of the lipophilic compound in a non-aqueous solvent to a turbulent zone in an aqueous solution of the polymer heated to a temperature above the boiling point of the non-aqueous solvent, to form the hydrophilic inclusion complexes.

There is an unmet need for cost effective and improved oral formulations which provide protection for active agents, particularly poorly water-soluble or amphiphilic active agents, against oxidation, heat, and enzymatic degradation in the upper gastrointestinal tract.

### SUMMARY OF THE INVENTION

The present invention provides methods for efficiently and reliably generating particles comprising non-covalent complexes comprising starch and active agents and having uniform particle size distributions in the microscale or nanoscale range. The particles are generated as a suspension in a liquid, which is readily converted to dry compositions. The compositions are particularly useful for oral delivery.

It is now disclosed that non-covalent complexes comprising starch and a low molecular weight poorly water-soluble or amphiphilic active agent form particles having a relatively uniform size below 50 μm that provide protection for the active agent against oxidation and heat. The non-covalent complexes release the active agent upon degradation by pancreatic amylases and therefore protect the active agent against degradation by enzymes present in the saliva and/or in the stomach.

It is now further disclosed that generating the particles comprising the non-covalent complexes of the present invention requires steps of feeding and homogenizing continuously the starch and the low molecular weight poorly water-soluble or amphiphilic active agent under high pressure in an aqueous solution, which steps enable producing the particles having a relatively uniform size below 50 μm. The methods of the present invention are rapid and cost effective as they utilize naturally occurring starch instead of amylose. As the methods of the present invention produce the non-covalent complexes by a continuous process which achieves high yields of the complexes, these methods are particularly advantageous over the currently available methods.

According to one aspect, the present invention provides a plurality of particles comprising non-covalent complexes comprising starch and at least one active agent, the particles having a uniform size below 50 μm, wherein the starch is other than vitamin B₁₂ coupled starch. According to specific embodiments, the non-covalent complexes are inclusion complexes.

According to some embodiments, the particles have a uniform size below 30 μm. According to further embodiments, the particles have a uniform size below 5 μm. According to yet further embodiments, the particles have a uniform size below 3 μm.

According to further embodiments, the starch is selected from the group consisting of unmodified natural starch and modified starch. According to yet further embodiments, the modified starch is selected from the group consisting of oxidized starch, esterified starch, cross linked starch, etherified starch, carboxymethylated starch, enzymatically modified starch, hydrolyzed starch, and heat treated starch. According to a particular exemplary embodiment, the starch is unmodified natural starch.

According to still further embodiments, the active agent is a low molecular weight agent selected from the group consisting of poorly water-soluble agents and amphiphilic agents. According to additional embodiments, the poorly water-soluble agent or amphiphilic agent is selected from the group consisting of drugs including, but not limited to, anticancer drugs, anti-inflammatory agents, antibacterial agents, peptides including, but not limited to, insulin, LHRH, calcitonin, growth factors, and antibacterial peptides, steroids, fatty acids, phytoestrogens including, but not limited to, isoflavones, vitamins including, but not limited to, vitamin A and vitamin D, prebiotic and probiotic compounds, nutrients, and flavors. According to yet further embodiments, the fatty acid is selected from the group consisting of saturated, unsaturated, monounsaturated, and polyunsaturated fatty acids. According to further embodiments, the monounsaturated or polyunsaturated fatty acid is selected from the group consisting of ω-3, ω-6, ω-9 fatty acids. According to a particular exemplary embodiment, the fatty acid is an ω-3 fatty acid.

According to further embodiments, the particles comprise non-covalent complexes consisting essentially of starch and an active agent, the particles having a uniform size below 50 μm, wherein the starch is other than B₁₂ coupled starch. According to yet further embodiments, the particles consist essentially of non-covalent complexes consisting essentially of starch and an active agent, the particles having a uniform size below 50 μm, wherein the starch is other than B₁₂ coupled starch. According to yet further embodiments, the particles have a uniform size below 30 μm. According to certain embodiments, the particles have a uniform size below 5 μm. According to specific embodiments, the particles have a uniform size below 3 μm.

According to another aspect, the particles comprising the non-covalent complexes of the present invention are formed after dissolution of the starch and the active agent in a solution having a basic pH and homogenization of the starch and the active agent dissolved in the solution having the basic pH with a solution having an acidic pH in a high-pressure dual feed homogenizer. According to yet further embodiments, the starch and the active agent are dissolved in two different or identical solutions having a basic pH. According to certain embodiments, the dissolution of the starch is performed at about 85° to about 95° C. for about 30 minutes to about 2 hours. According to further embodiments, the homogenization is a continuous homogenization.

According to a further aspect, the present invention provides a suspension comprising a plurality of particles comprising non-covalent complexes comprising starch and at least one active agent according to the principles of the present invention.

According to yet further aspect, the present invention provides a dry composition comprising as an active agent a plurality of particles comprising non-covalent complexes comprising starch and an active agent, the particles having a uniform size below 50 μm, wherein the starch is other than vitamin B₁₂ coupled starch, optionally comprising a pharmaceutically acceptable carrier.

According to some embodiments, the particles within the dry composition have a uniform size below 30 μm. According to additional embodiments, the particles within the dry composition have a uniform size below 5 μm. According to further embodiments, the particles within the dry composition have a uniform size below 3 μm.

According to further embodiments, the starch within the dry composition is selected from the group consisting of unmodified natural starch and modified starch. According to further embodiments, the modified starch within the dry composition is selected from the group consisting of oxidized starch, esterified starch, cross linked starch, etherified starch, carboxymethylated starch, enzymatically modified starch, hydrolyzed starch, and heat treated starch. According to a currently exemplary embodiment, the starch is unmodified natural starch.

According to yet further embodiments, the active agent within the dry composition is a low molecular weight agent selected from the group consisting of poorly water-soluble agents and amphiphilic agents. According to additional embodiments, the poorly water-soluble agent or amphiphilic agent within the dry composition is selected from the group consisting of drugs, peptides, fatty acids, phytoestrogens, steroids, prebiotic and probiotic compounds, vitamins, nutrients, anti-inflammatory agents, and antibacterial agents.

According to some embodiments, the dry composition can further comprise at least one additive selected from the group consisting of pH buffering agents, antioxidants, chelating agents, binders, lubricants, disintegrants, coloring agents, and flavoring agents.

According to further embodiments, the dry composition is selected from the group consisting of tablets, capsules, and pellets.

According to a further aspect, the present invention provides a method for preparing a suspension comprising a plurality of particles comprising non-covalent complexes comprising starch and an active agent, the method comprising the steps of:
(a) dissolving starch in a first solution having a basic pH to yield a starch solution;
(b) dissolving an active agent in a second solution having a basic pH to yield an active agent solution;
(c) mixing the starch solution of (a) and the active agent solution of (b) to yield a mixture of the starch and the active agent;
(d) feeding the mixture of (c) through a first opening into a high-pressure dual feed homogenizer;
(e) feeding a solution having an acidic pH through a second opening into the high-pressure dual feed homogenizer, wherein feeding the solution having the acidic pH is adjusted so as to produce a suspension having a pH in the range from about 4 to about 5 comprising a plurality of particles comprising non-covalent complexes comprising said starch and said active agent, the particles having a uniform size below 50 μm; and optionally
(f) drying the suspension of (e).

According to some embodiments, the starch that can be used for the preparation of the particles of the invention is selected from the group consisting of unmodified natural starch and modified starch. According to further embodiments, the modified starch useful for the preparation of said particles is selected from the group consisting of oxidized starch, esterified starch, cross linked starch, etherified starch, carboxymethylated starch, enzymatically modified starch, hydrolyzed starch, and heat treated starch. According to a particular exemplary embodiment, the starch is unmodified natural starch.

According to further embodiments, the active agent that can be used for the preparation of the particles of the present invention is a low molecular weight active agent selected from the group consisting of poorly water-soluble agents and amphiphilic agents. According to yet further embodiments, the poorly water-soluble agent or amphiphilic agent that can be used for the preparation of the particles of the present invention is selected from the group consisting of drugs, peptides, fatty acids, phytoestrogens, steroids, vitamins, prebiotic and probiotic compounds, anti-inflammatory agents, antibacterial agents, nutrients, and flavors. According to certain embodiments, the active agent is a fatty acid. According to specific embodiments, the active agent is a phytoestrogen.

According to additional embodiments, the solution having the basic pH is selected from the group consisting of a base of any pharmaceutically acceptable cation including, but not limited to, potassium hydroxide and sodium hydroxide. According to some embodiments, the solution having the basic pH is at a concentration in the range from about 0.01 M to about 1 M According to certain embodiments, the solution having the basic pH is potassium hydroxide at a concentration in the range from about 0.01 M to about 1 M. According to specific embodiments, potassium hydroxide is at a concentration from about 0.1 M to about 0.2 M. It is to be appreciated that the active agent must be resistant to the basic pH.

According to further embodiments, the step of dissolving the starch in the first solution is performed at a temperature of about 20° C. to about 95° C. for about 30 minutes to about 40 hours. According to certain embodiments, dissolving the starch in the first solution is performed at a temperature of 20° C. to 30° C. for 20 to 40 hours. According to specific embodiments, dissolving the starch in the first solution is performed at a temperature of 80° to 90° C. for 30 minutes to two hours. It is to be appreciated that the present invention encompasses shorter or longer dissolution time periods so long as the starch is dissolved.

According to yet further embodiments, the step of mixing the starch solution and the active agent solution is performed at a temperature of about 20° C. to about 95° C. According to certain embodiments, the step of mixing the starch solution and the active agent solution is performed at a temperature of about 30° to about 50° C.

According to further embodiments, the solution having the acidic pH is selected from the group consisting of an acid of any pharmaceutically acceptable anion including, but not limited to, hydrochloric acid, phosphoric acid, acetic acid, citric acid, and nitric acid. According to certain embodiments, the solution having the acidic pH is feed at a concentration in the range from about 0.01 M to about 1 M. According to certain embodiments, the solution having the acidic pH is phosphoric acid at a concentration in the range from about 0.01 M to about 1 M. According to specific embodiments, phosphoric acid is feed at a concentration from about 0.1 M to about 0.2 M.

According to additional embodiments, feeding the soluble mixture of (c) into the high-pressure dual feed homogenizer is performed at a pressure of about 1 Kpsi to about 100 Kpsi. According to certain exemplary embodiments, feeding the soluble mixture of (c) into the high-pressure dual feed homogenizer is performed at a pressure of about 10 Kpsi to about 30 Kpsi.

According to some embodiments, the particles have a uniform size below 30 μm. According to further embodiments, the particles have a uniform below 5 μm. According to certain embodiments, the particles have a uniform size below 3 μm.

According to further embodiments, drying is performed by freeze-drying, air-drying, or any drying method known in the art.

According to yet further aspect, the present invention provides methods for treating a disease in a subject comprising administering to the subject in need thereof a therapeutically effective amount of the dry composition according to the principles of the present invention, thereby treating the disease in the subject. According to some embodiments, the disease is cancer.

According to still further aspect, the present invention provides a method for providing a fatty acid to a subject comprising administering to the subject in need thereof an effective amount of the dry composition according to principles of the present invention, thereby providing the fatty acid to said subject. According to some embodiments, the fatty acid is selected from the group consisting of saturated, unsaturated, monounsaturated, and polyunsaturated fatty acids. According to further embodiments, the monounsaturated or polyunsaturated fatty acid is selected from the group consisting of ω-3, ω-6, and ω-9 fatty acids. According to an exemplary embodiment, the fatty acid is ω-3 fatty acid.

According to some embodiments, administering the dry composition of the invention is performed by oral administration.

According to another aspect, the present invention provides use of a plurality of particles comprising non-covalent complexes which comprise starch and an active agent according to the principles of the present invention for the preparation of a medicament for treating a disease.

According to another aspect, the present invention provides use of a plurality of particles comprising non-covalent complexes which comprise starch and an active agent according to the principles of the present invention for the preparation of a medicament for feeding a subject.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1 illustrates models of complex formation and structure. [A] Suggested mechanism of amylose-lipid complexes formation (Seneviratne and Biliaderis, J. Cereal Science, 1991. 13:129-143). [B] Suggested structure of amylose-fatty acid complexes as inferred from NMR and other studies (Kawada et al, 2004).

FIG. 2 shows XRD and ¹³C CP/MAS NMR spectra of complexes hosting stearic acid. On the left—XRD diffractograms verifying formation of V-amylose complexes type I and II. On the right—corresponding solid state ¹³C CP/MAS NMR spectra of the fatty acid in the complexes showing loss of signal resolution, indicating the fatty acid chain is less mobile in type I complexes.

FIG. 3 is a schematic representation of the process of the formation of non-covalent complexes of starch and active agent using high pressure dual feed homogenizer.

FIG. 4A-B show light scattering spectra of particle size distribution by volume of stearic acid—high amylose corn starch (HACS) mixture before homogenization (FIG. 4A) or after homogenization (FIG. 4B). Full lines represent light scattering spectra when the dissolution of the starch and fatty acid was performed at 85° C., while broken lines represent light scattering when the dissolution was performed at 25° C.

FIG. 5A-B show light scattering spectra of particle size distribution by volume of stearic acid—corn starch mixture before homogenization (FIG. 5A) or after homogenization (FIG. 5B). Full lines represent light scattering spectra when the dissolution of the starch and fatty acid was performed at 85° C., while broken lines represent light scattering when the dissolution was performed at 25° C.

FIG. 6A-B show light scattering spectra of particle size distribution by volume of stearic acid—waxy starch mixture before homogenization (FIG. 6A) or after homogenization (FIG. 6B). Full lines represent light scattering spectra when the dissolution of the starch and fatty acid was performed at 85° C., while broken lines represent light scattering when the dissolution was performed at 25° C.

FIG. 7 shows the release of a fatty acid from starch-fatty acid complexes after digestion with pancreatin.

FIG. 8 shows the release of a fatty acid from starch-fatty acid complexes as a function of time with pancreatin.

FIG. 9 shows the release of butyric acid from starch-fatty acid complexes after digestion by pancreatin.

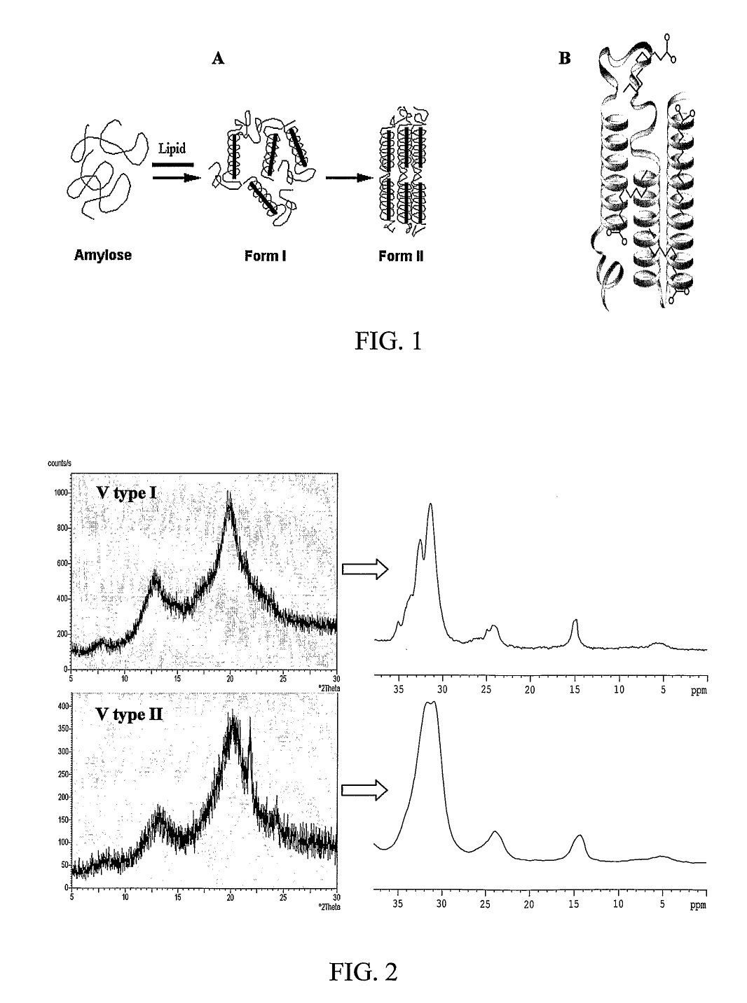

### DETAILED DESCRIPTION OF PREFERRED EMBODIMENTS

The present invention relates to non-covalent complexes comprising starch and a low molecular weight active agent selected from poorly water-soluble agents and amphiphilic agents, wherein the non-covalent complexes form particles having uniform size below 50 μm, particularly below 3 μm. It is to be understood that nowhere in the background art, complexes of starch and an active agent having such uniform size have been disclosed.

The starting material contemplated for use in the invention includes unmodified natural granular starches such as regular cereal, potato, and tapioca starch, and flours containing the same, waxy starch, high-amylose starch, and mixtures thereof. Full-fat starches, that is, starches which have not had a portion of the bound fat removed, are suitable for use herein.

Starch is a low-cost and abundant natural polymer composed of amylose and amylopectin. Amylose is essentially a linear polymer having a molecular weight in the range of 100,000-500,000, whereas amylopectin is a highly branched polymer having a molecular weight of up to several million. Common cornstarch (pearl) contains about 25% amylose and 75% amylopectin, waxy corn starches contain only amylopectin, and starches referred to as high-amylose starches contain up to 75% amylose.

The present invention also contemplates the use of modified starch. Modified starch includes, but is not limited to, oxidized starch, esterified starch, cross linked starch, etherified starch, carboxymethylated starch, enzymatically modified starch, hydrolyzed starch, and heat treated starch. It is to be understood that the present invention encompasses starch derivatives as known in the art.

The term “complex” as used herein is any physical combination of two or more discrete components. A complex includes, but is not limited to, a physical mixture and an inclusion complex.

The term “inclusion complex” as used herein refers to inclusion complexes wherein the active agent is surrounded by and entrapped within starch, and to partial inclusion complexes wherein the active agent is surrounded partially by starch. The starch presumably surrounds the hydrophobic regions of the active agent. It is to be appreciated that the non-covalent complexes of the present invention protect the active agent against oxidation, heat, and/or enzymatic degradation. As enzymes such as pancreatin that digest starch are present predominantly in the intestine, the release of the active agent occurs primarily at this location. Thus, the non-covalent complexes of the present invention protect the active agent against degradation by enzymes present in the saliva and/or stomach. According to the principles of the present invention, the non-covalent complex is preferably an inclusion complex.

The term “non-covalent complex” as used herein refers to a complex in which the bonds between the components of the complex are non-covalent bonds, i.e., weak bonds such as H-bonds and Van der Waals forces.

The active agent is preferably a low molecular weight agent selected from the group consisting of poorly water-soluble agents and amphiphilic agents.

The term “poorly water-soluble” agent as used herein refers to a compound that typically has solubility in water below 1 gr/30 ml at room temperature. The present invention encompasses water-insoluble agents which are compounds that typically have solubility in water of less that 1 gr/10,000 ml at room temperature.

The term “amphiphilic” agent as used herein refers to an agent having a hydrophobic portion and a hydrophilic portion

The poorly water-soluble agents and amphiphilic agents that constitute the non-covalent complexes of the present invention include, but are not limited to, drugs, peptides, fatty acids, steroids, phytoestrogens, pro-biotic compounds, and vitamins.

Drugs that can constitute the non-covalent complexes of the present invention include, but are not limited to, anti-infectives such as antibacterial agents, antiviral agents, analgesics and analgesic combinations, anesthetics, anti-arthritics, anti-asthmatic agents, anticonvulsants, anti-depressants, anti-diabetic agents, anti-diarrhea agents, antihistamines, anti-inflammatory agents, anti-migraine preparations, anti-motion sickness preparations, anti-nauseants, anti-neoplastics, anti-parkinsonism drugs, antipruritics, antipsychotics, antipyretics, antispasmodics including gastrointestinal and urinary, anticholinergics, sympathomimetics, xanthine derivatives, cardiovascular preparations including calcium channel blockers, beta-blockers, antiarrhythmics, antihypertensives, diuretics, vasodilators including general, coronary, peripheral and cerebral vasodilators, central nervous system stimulants, cough and cold suppressants, decongestants, hypnotics, immunosuppressives, muscle relaxants, parasympatholytics, parasympathomimetics, psychostimulants, sedatives, tranquilizers, and anticancer drugs.

Anticancer drugs that can be used as constituents of the non-covalent complexes of the present invention include, but are not limited to, cytotoxic, cytostatic and antiproliferative drugs such as are known in the art, exemplified by such compounds as:
Alkaloids: Docetaxel, Etoposide, Irinotecan, Paclitaxel, Teniposide, Topotecan, Vinblastine, Vincristine, Vindesine.
Alkylating agents: Busulfan, Improsulfan, Piposulfan, Benzodepa, Carboquone, Meturedepa, Uredepa, Altretamine, triethylenemelamine, Triethylenephosphoramide, Triethylenethiophosphoramide, Chlorambucil, Chloranaphazine, Cyclophosphamide, Estramustine, Ifosfamide, Mechlorethamine, Mechlorethamine Oxide Hcl, Melphalan, Novemebichin, Perfosfamide Phenesterine, Prednimustine, Trofosfamide, Uracil Mustard, Carmustine, Chlorozotocin, Fotemustine, Lomustine, Nimustine, Semustine Ranimustine, Dacarbazine, Mannomustine, Mitobronitol, Mitolactol, Pipobroman, Temozolomide.
Antibiotics and analogs: Aclacinomycins, Actinomycins, Anthramycin, Azaserine, Bleomycins, Cactinomycin, Carubicin, Carzinophilin, Cromomycins, Dactinomycins, Daunorubicin, 6-Diazo-5-oxo-L-norleucine, Doxorubicin, Epirubicin, Idarubicin, Menogaril, Mitomycins, Mycophenolic Acid, Nogalamycine, Olivomycins, Peplomycin, Pirarubicin, Plicamycin, Porfiromycin, Puromycine, Streptonigrin, Streptozocin, Tubercidin, Zinostatin, Zorubicin.
Antimetabolites: Denopterin, Edatrexate, Methotrexate, Piritrexim, Pteropterin, Tomudex, Trimetrexate, Cladridine, Fludarabine, 6-Mercaptopurine, Pentostatine Thiamiprine, Thioguanine, Ancitabine, Azacitidine, 6-Azauridine, Carmofur, Cytarabine, Doxifluridine, Emitefur, Floxuridine, Fluorouracil, Gemcitabine, Tegafur;
Platinum complexes: Caroplatin, Cisplatin, Miboplatin, Oxaliplatin;
Others: Aceglatone, Amsacrine, Bisantrene, Defosfamide, Demecolcine, Diaziquone, Eflornithine, Elliptinium Acetate, Etoglucid, Etopside, Fenretinide, Gallium Nitrate, Hdroxyurea, Lonidamine, Miltefosine, Mitoguazone, Mitoxantrone, Mopidamol, Nitracrine, Pentostatin, Phenamet, Podophillinic acid 2-Ethyl-Hydrazide, Procarbazine, Razoxane, Sobuzoxane, Spirogermanium, Teniposide Tenuazonic Acid, Triaziquone, 2,2′,2″-Trichlorotriethylamine, Urethan.

Peptides that can constitute the non-covalent complexes of the present invention have preferably a molecular weight below 10 kDa. More preferably, the peptides have a molecular weight below 6 kDa. Examples of peptides include, but are not limited to, insulin, erythropoietin, epidermal growth factor, nerve growth factor, transforming growth factors, calcitonin, parathyroid hormone, glucagon, atrial natriuretic factor, bombesin, and LHRH, fragments, and biologically active analogs thereof.

Fatty acids that can constitute the non-covalent complexes of the invention include saturated, monounsaturated and polyunsaturated fatty acids including ω-3, ω-6, and ω-9 fatty acids. Examples of the fatty acids that can constitute the non-covalent complexes of the invention include, but are not limited to, decanoic acid, undecanoic acid, dodecanoic acid, myristic acid, palmitic acid, stearic acid, arachidic acid, lignoceric acid, palmitoleic acid, oleic acid, linoleic acid, α linolenic acid, arachidonic acid, eicopentaenoic acid, and docosahexaenoic acid.

Phytoestrogens are non-steroidal compounds found in a variety of plants which exert estrogenic effects in animals. Phytoestrogens consist of a number of classes including isoflavones, coumestans, lignans and resorcylic acid lactones. The class of isoflavones consists of among others genistein (4′,5,7-trihydroxyisoflavone), daidzein (4′,7-dihydroxyisoflavone), equol, glycitein, biochanin A, formononetin, and O-desmethylangolesin. The isoflavones genistein and daidzein are found almost uniquely in soybeans. When present in the plant the isoflavones are mainly in a glucoside form, i.e. attached to a sugar molecule. Isoflavones in this glucoside form can be deconjugated to yield isoflavones in a so-called aglycone form, which is the biologically more active form of isoflavones and which is absorbed faster and to a greater extent in the human gut than isoflavones in the glucoside form. Thus, the present invention encompasses the glucoside form and the aglycone form of isoflavones.

It is to be understood that the generation of the non-covalent complexes of the present invention excludes the use of organic solvents. Unexpectedly, the present invention provides methods for generating non-covalent complexes wherein a plurality of the non-covalent complexes forms particles having a uniform size below 50 μm, which methods do not require the addition of any organic solvent, but do require dual feeding of basic and acidic solutions under high pressure homogenization. The methods of the present invention enable achieving a homogenous suspension comprising nano- or micro-particles of the non-covalent complexes of the invention.

The term “particle” as used herein refers to a globular cluster, a rod like cluster, and the like made of two or more non-covalent complexes wherein the non-covalent complexes comprise starch and an active agent. According to the principles of the present invention, the particles comprising said non-covalent complexes have a relatively uniform size below 50 μm. The terms nano- or micro-capsules refer to the nano- or micro-particles indicated herein above and are used interchangeably throughout the specification and claims.

The terms “uniform size” or “uniform size distribution” as used herein mean that the particles have size distribution such that D₉₀ is less than about 50 μm (90% of particles are smaller than the D₉₀ value) in the longest dimension of the particles. Thus, the particles of the present invention have a D₉₀ not exceeding 50 μm. According to certain embodiments, the particles have a D₉₀ not exceeding 30 μm, not exceeding 5 μm, or not exceeding 3 μm. The particle sizes stipulated herein and in the claims refer to particle sizes determined by light scattering.

The term “about” as used herein refers to a deviation of ±10% of any value indicated such as diameter, pH, temperature, and the like.

The present invention provides a method for preparing a suspension comprising a plurality of particles comprising non-covalent complexes comprising starch and an active agent, the method comprises the steps of:
(a) dissolving starch in a first solution having a basic pH to yield a starch solution;
(b) dissolving an active agent in a second solution having a basic pH to yield an active agent solution;
(c) mixing the starch solution of (a) and the active agent solution of (b) to yield a mixture of the starch and the active agent;
(d) feeding, optionally continuously, the mixture of (c) and a solution having an acidic pH into a high-pressure dual feed homogenizer, wherein feeding the solution having an acidic pH is adjusted so as to produce a suspension having a pH in the range of about 4 to about 5 comprising a plurality of particles comprising non-covalent complexes which comprise said starch and said active agent, the particles having a uniform size below 50 μm; and optionally
(e) drying the suspension of (d).

It is to be understood that while steps (a) and (b) can be performed separately, these two steps can be combined. Accordingly, the present invention provides a method for preparing a suspension comprising a plurality of particles comprising non-covalent complexes which comprise starch and an active agent, the method comprises the steps of:
(a) dissolving starch and an active agent in a solution having a basic pH to yield a mixture of starch and active agent;
(b) feeding, optionally continuously, the mixture of (a) and a solution having an acidic pH into a high-pressure dual feed homogenizer, wherein feeding the solution having the acidic pH is adjusted so as to produce a suspension having a pH in the range of about 4 to about 5 comprising a plurality of particles which comprise non-covalent complexes comprising said starch and said active agent, the particles having a uniform size below 50 μm; and optionally
(c) drying the suspension of (b).

It is to be understood that the active agents that can be used in the present invention should be resistant to basic pHs so that the biological activity of these agents is maintained after the non-covalent complexes have been formed.

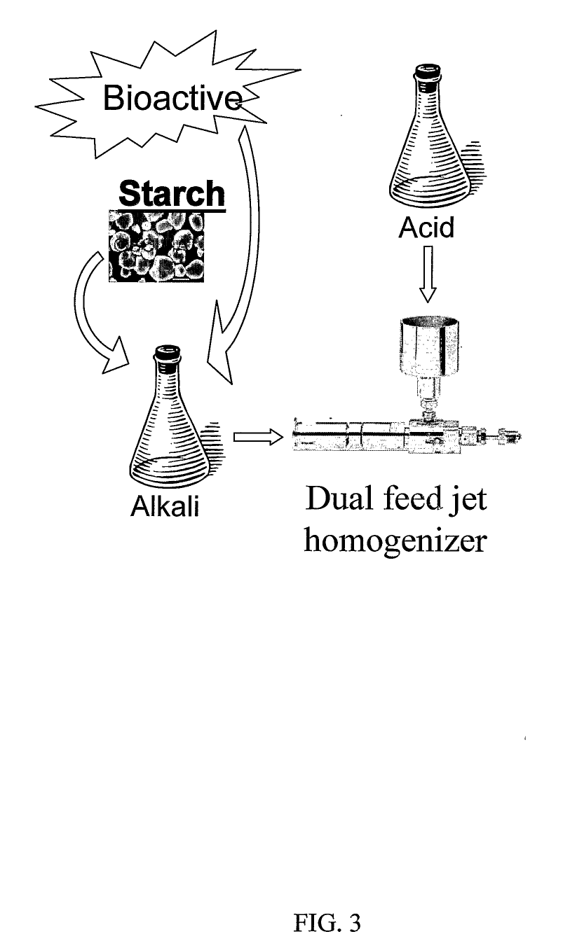

The present invention encompasses any high pressure dual feed homogenizer known in the art, such as for example Micro DeBee homogenizer (see for example U.S. Pat. No. 6,255,393, the content of which is incorporated by reference as if fully set forth herein). Thus, according to the present invention, feeding the mixture of starch and active agent is performed through a first opening of the high pressure dual homogenizer, while feeding the solution having the acidic pH is performed through a second opening of the homogenizer (see FIG. 3). Typically, the first opening is a principal opening of the homogenizer through which the mixture of the invention is feed, while the second opening is oriented vertically to the first opening and the feeding of the acidic solution through the second opening is performed by vacuum created by the well known ventury effect. Accordingly, the present invention provides a method for preparing a suspension comprising a plurality of particles comprising non-covalent complexes which comprise starch and an active agent, the method comprises the steps of:
(a) dissolving starch in a first solution having a basic pH to yield a starch solution;
(b) dissolving an active agent in a second solution having a basic pH to yield an active agent solution;
(c) mixing the starch solution of (a) and the active agent solution of (b) to yield a mixture of the starch and the active agent;
(d) feeding, optionally continuously, the mixture of (c) through a first opening of a high-pressure dual feed homogenizer;
(e) feeding, optionally continuously, a solution having an acidic pH through a second opening of the high-pressure dual feed homogenizer, wherein feeding the solution having the acidic pH is adjusted so as to produce a suspension having a pH in the range of about 4 to about 5 comprising a plurality of particles comprising non-covalent complexes which comprise said starch and said active agent, the particles having a uniform size below 50 μm; and optionally
(f) drying the suspension of (e).

The present invention encompasses non-covalent complexes comprising starch and at least one active agent. However, in currently exemplary embodiments, the non-covalent complexes consist essentially of starch and an active agent.

According to the present invention, dissolution of starch can be performed at room temperature up to 95° C. for 30 minutes to 40 hours. It should be understood that the present invention discloses that dissolution of the starch in a basic solution at high temperatures, e.g., 85° to 95° C. for 30 minutes to two hours followed by mixing the dissolved starch with an active agent solution to yield a mixture of starch and active agent, and then homogenization of the mixture with acidic solution in a high-pressure dual feed homogenizer resulted in the formation of smaller particles than if the dissolution of the starch was performed at room temperature for 24 hours, for example. Thus, according to certain preferred embodiments, starch dissolution is performed at 85° to 95° C. for 30 minutes to two hours to produce particles of uniform small particle size.

### Pharmaceutical or Nutraceutical Compositions

The present invention provides pharmaceutical or nutraceutical compositions comprising the particles of the present invention and optionally a pharmaceutically acceptable carrier. For sake of brevity, the term “composition” is used exchangeably with pharmaceutical or nutraceutical compositions.

The term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic or nutraceutical compound is administered. Such pharmaceutical carriers can be sterile liquids, such as water, aqueous dextrose, and oils, including those of petroleum, animal, vegetable or synthetic origin, such as peanut oil, soybean oil, mineral oil, sesame oil and the like, polyethylene glycols, glycerin, propylene glycol or other synthetic solvents. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like. The composition, if desired, can also contain minor amounts of wetting or emulsifying agents, pH buffering agents such as acetates, citrates or phosphates; antibacterial agents such as benzyl alcohol or methyl parabens; antioxidants such as ascorbic acid or sodium bisulfite; chelating agents such as ethylenediaminetetraacetic acid; binders; lubricants; disintegrants; coloring agents; and flavoring agents.

As used herein, “binders” are agents used to impart cohesive qualities to a powdered material. Binders, or “granulators” as they are sometimes known, impart a cohesiveness to a tablet formulation, which insures the tablet remaining intact after compression, as well as improving the free-flowing qualities by the formulation of granules of desired hardness and size. Materials commonly used as binders include starch; gelatin; sugars, such as sucrose, glucose, dextrose, molasses, and lactose; natural and synthetic gums, such as acacia, sodium alginate, extract of Irish moss, panwar gum, ghatti gum, mucilage of isapol husks, carboxymethylcellulose, methylcellulose, polyvinylpyrrolidone, Veegum, microcrystalline cellulose, microcrystalline dextrose, amylose, and larch arabogalactan, and the like.

As used herein, “lubricants” are materials that perform a number of functions in tablet manufacture, such as improving the rate of flow of the tablet granulation, preventing adhesion of the tablet material to the surface of the dies and punches, reducing interparticle friction, and facilitating the ejection of the tablets from the die cavity. Commonly used lubricants include talc, magnesium stearate, calcium stearate, stearic acid, and hydrogenated vegetable oils. Typical amounts of lubricants range from about 0.1% by weight to about 5% by weight.

As used herein, “disintegrants” are substances that facilitate the breakup or disintegration of tablets after administration. Materials serving as disintegrants have been chemically classified as starches, clays, celluloses, algins, or gums. Other disintegrators include Veegum HV, methylcellulose, agar, bentonite, cellulose and wood products, natural sponge, cation-exchange resins, alginic acid, guar gum, citrus pulp, cross-linked polyvinylpyrrolidone, carboxymethylcellulose, and the like.

As used herein, “coloring agents” are agents that give tablets a more pleasing appearance, and in addition help the manufacturer to control the product during its preparation and help the user to identify the product. Any of the approved certified water-soluble FD&C dyes, mixtures thereof, or their corresponding lakes may be used to color tablets. A color lake is the combination by adsorption of a water-soluble dye to a hydrous oxide of a heavy metal, resulting in an insoluble form of the dye.

As used herein, “flavoring agents” vary considerably in their chemical structure, ranging from simple esters, alcohols, and aldehydes to carbohydrates and complex volatile oils. Synthetic flavors of almost any desired type are now available.

The pharmaceutical or nutraceutical compositions of the present invention are preferably dry compositions. The dry compositions can take the form of tablets, capsules, powders, sustained-release formulations and the like. However, the present invention encompasses liquid or semi-liquid compositions. The liquid or semi-liquid compositions can take the form of solutions, suspensions, emulsions, and gels. It should therefore be appreciated that compositions comprising the particles of the present invention formulated in a liquid or semi-liquid form are included within the scope of the invention.

The preparation of dry compositions which contain an active component is well understood in the art, for example by mixing, granulating, or tablet-forming processes. The active agent is often mixed with excipients which are pharmaceutically acceptable and compatible with the active agent. For oral administration, the particles of the present invention can be mixed with additives customary for this purpose, such as an inert pharmaceutically acceptable carrier, and converted by customary methods into suitable forms for administration, such as tablets, coated tablets, hard or soft gelatin capsules

### Uses of the Non-Covalent Complexes

The present invention provides methods for treating a disease in a subject comprising administering to the subject in need thereof a composition comprising a therapeutically effective amount of the particles comprising the non-covalent complexes of the present invention. The composition can be formulated in a dry, liquid, or semi-liquid form. According to a certain aspect, the method for treating a disease in a subject comprises a step of administering to the subject the dry composition of the invention.

As used herein, the term “treating” means remedial treatment, and encompasses the terms “reducing”, “suppressing” “ameliorating” and “inhibiting”, which have their commonly understood meaning of lessening or decreasing.

A “therapeutically effective amount” of the particles comprising the non-covalent complexes is that amount of the particles which is sufficient to provide a beneficial effect to the subject to which the complex is administered.

Diseases that can be treated by the compositions of the present invention include, but are not limited to, hormone-mediated diseases, inflammatory diseases, autoimmune diseases, infections, neurodegenerative diseases, and cancer.

Cancer that can be treated with the pharmaceutical compositions of the invention include malignant and metastatic conditions including, but not limited to, solid tumors such as sarcoma, carcinoma, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor leiomydsarcoma, rhabdomyosarcoma, colon carcinoma, pancreatic cancer, breast cancer, ovarian cancer, prostate cancer, squamous cell carcinoma, basal cell carcinoma, adenocarcinoma sweat gland carcinoma, sebaceous gland carcinoma, papillary carcinoma, papillary adenocarcinoma, cystadenocarcinoma, medullary carcinoma, bronchogenic carcinoma, renal cell carcinoma, hepatoma bile duct carcinoma, choriocarcinoma, seminoma, embryonal carcinoma, Wilms' tumor cervical cancer, testicular tumor, lung carcinoma, small cell lung carcinoma, bladder carcinoma, epithelial carcinoma, glioma, astrocytoma, medulloblastoma, craniopharyngioma, ependymoma, Kaposi's sarcoma, pinealoma, hemangioblastoma, acoustic neuroma, oligodendroglioma, menangioma, melanoma, neuroblastoma, and retinoblastoma. The pharmaceutical compositions of the invention can also be used for the treatment of non-solid tumors including, but not limited to, leukemia and lymphoma.

The present invention further provides methods for providing a fatty acid to a subject in need thereof.

According to some embodiments of the invention, the subject is a mammal, preferably a human. It should be therefore understood that the pharmaceutical or nutraceutical compositions of the present invention can be administered to a newborn, child, adult, and a chronically ill subject.

The pharmaceutical or nutraceutical compositions of the invention can be administered in combination with any other conventional therapy.

### Example 1

### Effect of Encapsulation of Saturated, Omega 3, and Polyunsaturated Fatty Acids by Amylose

The aim of these studies was to monitor the effects of guest molecular structure on nano-capsule properties via host-guest interactions and the feasibility of such nanoencapsulation. To that end, different fatty acids having different conformations were used as ligands:unsaturated 18:0 stearic acid (SA), cis-cis-18:2 linoleic acid (LA), cis-trans-18:2 conjugated linoleic acid (CLA), 20:4 arachidonic acid (AA), 20:5 eicosapentaenoic acid (EPA) as well as 22:5 docosahexadecanoic acid (DHA). Amylose-lipid complexes produced with each of these fatty acids (FAs) was studied by DSC to determine thermostability, XRD to determine complex crystallinity, Nuclear Magnetic Resonance (NMR) techniques to study the conformation, mobility and the arrangement of the ligands in the hydrophobic pocket, and AFM/NSOM to characterize supramolecular structure.

### Experimental

Amylose-fatty acid mixtures (10:1 w/w) were dissolved in DMSO at 90° C., then rapidly diluted (1:20 w/w) into water and allowed to complex for 15 min in a water bath. Complexes formed were washed, separated by centrifugation and freeze dried into a fine powder that was analyzed by DSC and XRD to estimate the degree of V-I and V-II complex form formation. Solution and solid state ¹³C NMR were also used to follow complexation in solution and decipher the crystal structure at the solid state.

### Results

X-ray diffraction of the amylose-fatty acids mixture powders proved to yield typical diffractograms pointing to the successful formation of complexes hosting SA, LA, CLA, AA and DHA. Functionality of the nanocapsules was evaluated by testing the stability of CLA to heat and oxidation as well as the enzymatic release profile (Lalush et al, 2005). ¹H NMR of amylose-SA complexes showed no evidence of complexation in the DMSO while rapidly after dilution into double distilled water (dilution ratio of 1:20) most of the SA signals disappeared, indicating loss of mobility attributed to complexation. ¹³C CP/MAS NMR of complexes hosting SA showed the presence of polymorph I and II, which differ, in the X ray diffraction (as previously mentioned in literature) and in the mobility of the encapsulated FA. Furthermore, ¹³C CP/MAS NMR spectra of complexes hosting LA and CLA pointed to differences in mobility of both the FA and the amylose, especially in C2 and C3,5, indicating lower mobility of the glucopyranose, which might affect amylose digestibility by amylases.

### Example 2

### Alternative Methods for the Production of Amylose- or Starch-Fatty Acid Mixtures

The aim of these experiments was to monitor functional and structural properties of amylose- or starch-fatty acid complexes produced by different complexation processes.

### Experimental

Four complexation processes were tested:
1. Amylose-lipid complexes were produced via DMSO as follows: amylose-fatty acid mixtures (10:1 w/w) were dissolved in DMSO at 90° C., then rapidly diluted (1:20 w/w) into water and allowed to complex for 15 min in a water bath. Complexes formed were washed, separated by centrifugation and freeze dried into a fine powder;
2. Amylose-lipid complexes were produced by standard acidification reaction as follows: amylose and fatty acid were separately dissolved in an alkali solution (0.1 M KOH), mixed, and then the pH was lowered to about 4.7, leading to a 24 hr crystallization step at either 90° C. or 30° C.;
3. Food grade high amylose corn starch (HACS) and stearic acid, the stearic acid used as a model guest fatty acid, were subjected to acidification reaction in the presence of sodium hydroxide and phosphoric acid to form V-amylose; and
4. An adaptation of the common batch production process was performed to enable continuous production of nanocapsules. Thus, one liter of HACS and stearic acid, both dissolved in 0.1 M sodium hydroxide, were used to form V-amylose complexes via a continuous dual feeding of the mixture solution and phosphoric acid using Micro DeBEE Laboratory Homogenizer (Electro-hydraulically operated high pressure homogenizer; FIG. 3) operating at high pressures (˜1000 bar).

Immediately after production, all complexes were separated and molecularly characterized to verify the successful formation of V-amylose. To enable better resolution of the NMR studies, ¹³C labeled SA (fully labeled SA and C2 labeled SA) was used in the DMSO or KOH/HCl acidification methods together with fully deuterated solvents and reagents.

### Results

Complexes produced via all four methods proved to yield typical V-amylose x-ray diffraction. Functionality studies (i.e., thermal/oxidative stability and enzymatic release) indicated that nanoencapsulation of CLA provides protection from highly acidic conditions and oxidation while allowing CLA release by pancreatic alpha-amylase. ¹³C CP/MAS NMR of complexes hosting unlabeled SA made by DMSO or KOH/HCl processes showed differences in mobility of both SA and the amylose, especially in the C2 and C3,5 and C(4)-O—C(1) bonds which are the bonds hydrolyzed by many amylases. The co-extrusion process proved to enable very fast and continuous production of V-amylose complexes hosting stearic acid, as indicated by the x-ray diffraction pattern of the powders produced.

### Example 3

### Complex Formation of Fatty Acids and Starch Under Different Dissolution Temperatures

Inclusion of the fatty acids in V-amylose nanocapsules was carried out based on a method previously described (Lalush et al, 2005, Biomacromolecules, 6: 121-130, 2005). Basically, 6 g of the indicated starch, i.e., high amylase corn starch (HASC), corn starch, and waxy starch, were dissolved in 400 ml 0.1 M KOH solution, either at room temperature for 24 hours or at ˜85° C. for an hour. Resulting solution was then mixed with 600 ml of 0.1 M KOH containing 0.45 g stearic acid at similar temperature (25° C. or ˜85° C., respectively). The resulting 1 liter of alkali mixture was then homogenized with 0.1-0.2 M phosphoric acid to yield a cloudy solution at a pH of ˜5 by adjusting the flow rate of the acid to the homogenizer (each starch and operating pressure level required a different acidic concentration to achieve proper outlet pH). The high pressure homogenization was performed in Micro DeBee homogenizer purchased from BEE international, in which alkali solutions were pressured through a nozzle at an operating pressure of 25 Kpsi with acid solution (FIG. 3). Approximately 200 ml of the resulting suspension was used for particle size analysis and the remainder of the each suspension was centrifuged (4750 rpm, 20 min, 20° C.) and then freeze-dried and pulverized into a fine powder for crystal characterization by X-ray diffraction.

### Particle Size Analysis by Light Scattering

Suspensions produced during the complexation process were analyzed by light scattering to monitor the particle size distribution of the resulting complexes in the native suspension state. Measurements were obtained by measuring the Laser scattering of the suspensions over 2 sequential periods of 90 seconds in a LS230 coulter counter equipped with PIDS module (Polarized Intensity Differential Scattering module) using 3 wavelengths and 2 polarization directions. Analysis of the scattered light was based on the general fraunhofer optical model with water as solvent and the data was analyzed by the number of particles, their surface area and their volume.

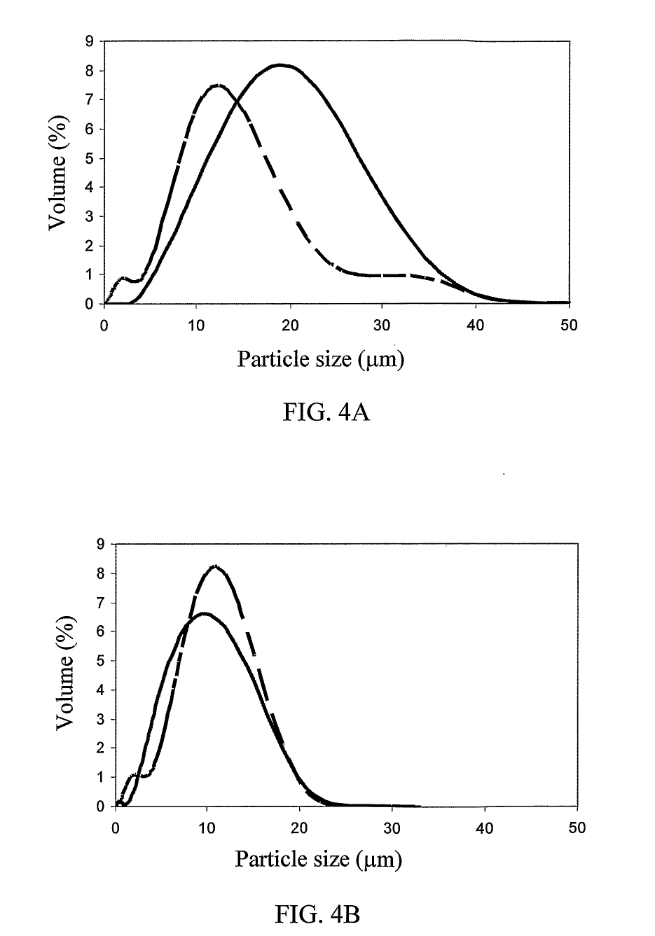

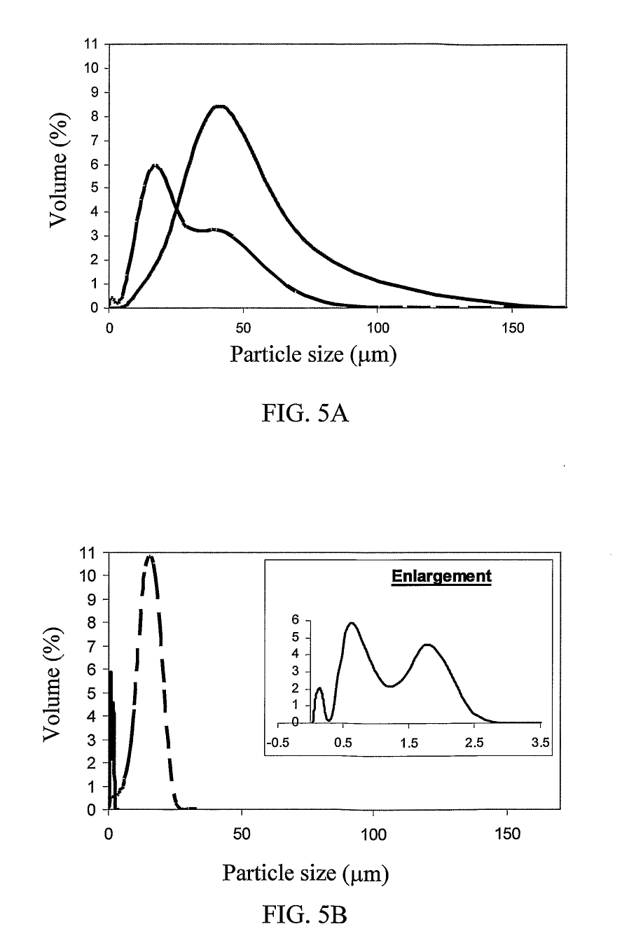

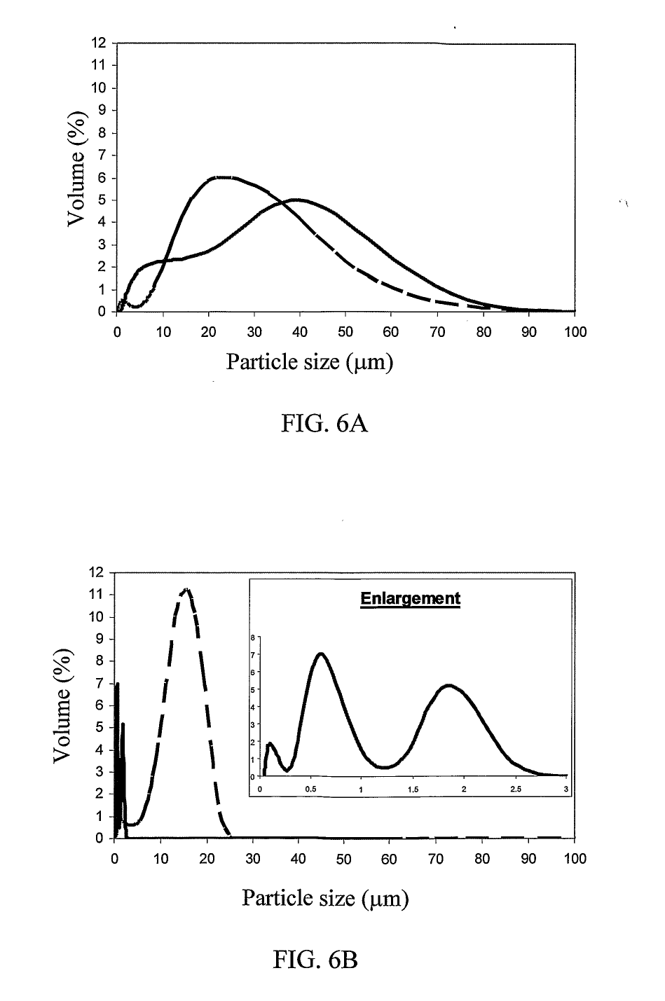

The results indicate that homogenization under high pressure of the fatty acid-starch mixtures resulted in the formation of particles having uniform diameter (FIGS. 4B, 5B, and 6B) as compared to the diameter of the particles before homogenization (FIGS. 4A, 5A, and 6A). The particle diameter was significantly smaller after homogenization (FIGS. 4B, 5B, and 6B) than before homogenization (FIGS. 4A, 5A, and 6A). In addition, dissolution of the starch and fatty acid at 85° C. and subsequent homogenization with acidic acid under high pressure resulted in the formation of particles having even smaller diameter than that of the particles dissolved at room temperature and then homogenized under the same conditions (FIGS. 5B and 6B).

### Example 4

### Release of Fatty Acid from Starch-Fatty Acid Complexes by Pancreatin

Lyophilized powders of the starch-fatty acid complexes prepared in Example 3 herein above were subjected to enzymatic digestion by pancreatic amylases in order to evaluate their guest content by Gas Chromatography (GC). The analysis was carried out in two steps: first the sample was digested and extracted and then stearic acid content was determined by GC analysis based on a calibration curve. As negative controls, lyophilized powders of the complexes were subjected to the same conditions as the pancreatin-treated complexes but without pancreatin. Positive controls contained double amount of pancreatin.

### Preparation of Pancreatin Solution

Physiological conditions were simulated by PBS (Phosphate Buffer Saline, pH=6.9) composed of 1.571 g Na₂HPO₄ and 1.23 g of KH₂PO₄ mixed in 800 ml of distilled water with 65 ml of NaCl solution (0.9 g NaCl in 100 ml distilled water) followed by pH adjustment to 6.9 (using 1 M NaOH) and volume brought up to 1 L with distilled water. Pancreatin solutions were prepared by dissolving 0.1783 g (for the sample test) or 0.3566 g (for the positive controls of double amount of pancreatin) in 20 ml of PBS solution at room temperature for 30 minutes. The solutions were then centrifuged (1500 rpm, 20° C., 5 min) and the supernatant was collected to be used as a pancreatin solution.

### Digestion

Fifty mg of each starch-FA complex were dissolved in 2.5 ml of pancreatin solution in a glass vial. The samples and the controls were then left in a 37° C. shaking bath for 24 hours. All samples and controls were done in duplicates.

### Extraction

Stearic acid was extracted by analytical grade hexane. At the end of 24 hour incubation in the bath, 2.5 ml of hexane were added to each vial and then vortexed for 30 seconds. The upper hexane phase was kept and a second extraction was done with 2.5 ml of hexane. Finally, hexane was evaporated by a gentle flow of N₂ and the vial was kept at −20° C. until further analysis.

### Quantification by GC

Frozen glass vials were acclimated to room temperature and then re-suspended in filtered ethanol. Stearic acid content was determined by injecting 0.1 μl of a sample into a GC (Hewlett-Packard GCD system HP 5890) equipped with an HP-Innowax capillary column [30 m×0.32 mm (i.d.) with 0.25 μm film thickness; HP]. The temperature programming was as follows: 120° C. for 1 min, then increments of 10° C./min to 250° C., and finally 250° C. for 2 min. Inlet and detector temperatures were 250° C. The nitrogen carrier gas flow rate was 2.4 ml/min, hydrogen flow to the detector was 25 ml/min, airflow was 400 ml/min, and the flow of nitrogen makeup gas was 45 ml/min. Peaks were identified by comparison with standards for stearic acid used as a calibration curve. Each sample was injected twice thus each experiment point was analyzed four times (2 injections×2 samples per point).

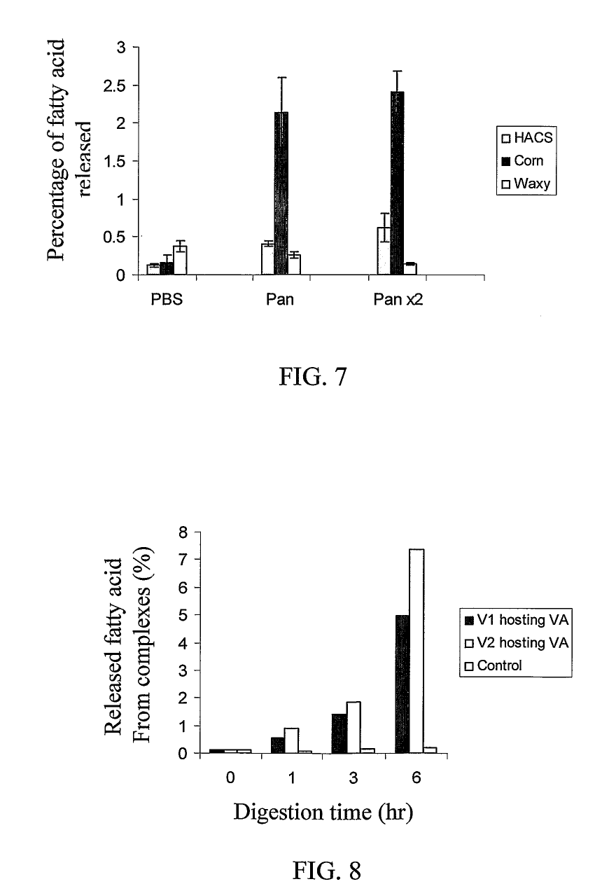

The results shown in FIG. 7 indicate that stearic acid was released from starch—stearic acid complexes after digestion by pancreatin. The highest release was observed from HACS—stearic acid complexes.

FIG. 8 shows the release of stearic acid from 2 different crystalline polymorphs of V-amylose complexes after digestion for periods of time with pancreatin. Controls were done in pure PBS solution without pancreatin.

### Example 5

### Encapsulation of Allicin

The aim of this experiment was to determine whether encapsulation of highly sensitive bioactive agents can be used as a technological tool for the targeted and controlled delivery of these agents to the lower GI.

### Experimental

Commercially available HACS was used to encapsulate the anti-carcinogenic nutraceutical allicin (extracted from garlic) that shows high sensitivity to the conditions of the stomach. HACS was dissolved into an alkali solution (0.1 M NaOH at 90° C.), then allicin was dissolved in a similar solution and mixed with HACS solution. The pH of the mixture was then lowered to moderate acidic values using 0.1 M phosphoric acid, crystallized for 24 hrs in a water bath kept at 30° C., and the resulting complexes were then separated, freeze dried and analyzed.

### Results

Evaluation of the targeted and controlled release of allicin from the complexes is achieved by determining the bioactivity of the supernatant of complexes dissolved in PBS against cancer cells before and after 2 hr enzymatic digestion of the starch with pancreatin. The supernatants are also tested by HPLC to detect the presence of allicin and/or its derivatives. The results indicated that pancreatin treated complexes liberated allicin into the PBS medium, whereas non-treated complexes showed negligible levels of allicin in the hydrating PBS buffer.

### Example 6

### Encapsulation of a Probiotic Compound

The aim of this experiment was to determine whether encapsulation of butyric acid, a pro-biotic compound, can be used as a technological tool for the targeted and controlled delivery of this agent to the lower GI.

### Experimental

Commercially available HACS was used to encapsulate butyric acid that shows high sensitivity to the conditions of the stomach. HACS was dissolved into an alkali solution (0.1 M NaOH at 90° C.), then butyric acid was dissolved in a similar solution and mixed with HACS solution. The alkali pH of the mixture was then lowered to moderate acidic values of ˜5 using 0.1 M phosphoric acid, crystallized under gentle stirring in a flask for 24 hrs in a water bath kept at 30° C., and the resulting complexes were then separated, freeze dried and analyzed.

### Results

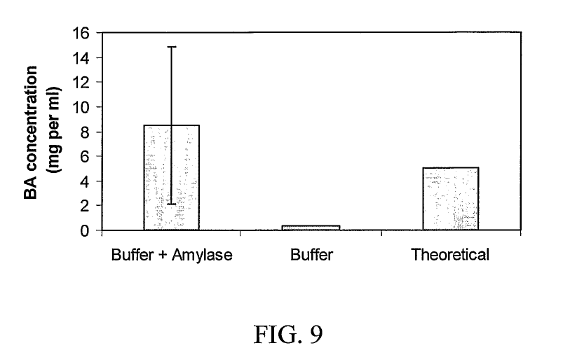

The release of butyric acid from the complexes was tested by GC. The results indicated that pancreatin treated complexes liberated butyric acid into the PBS medium, whereas non-treated complexes showed negligible levels of butyric acid in the hydrating PBS buffer (FIG. 9). The theoretical histogram shows the calculated predicted content of butyric acid in the complex—which resembles the concentration of butyric acid being released by enzymatic digestion.

### Example 7

### Encapsulation of a Phytoestrogen

The aim of this experiment was to determine whether encapsulation of genistin, a pyhtoestrogen, can be used as a technological tool for the targeted and controlled delivery of this agent to the lower GI. Genistin as other phytoestrogens are also a source for flavors in food products.

### Experimental

Commercially available HACS was used to encapsulate genistin. HACS was dissolved into an alkali solution (0.1 M KOH at 85° C.) and the solution was cooled to 30° C. Genistin (Solbar isoflavones extract 40 S) was added at room temperature and the solution was entered into the homogenizer at 18-23 Kpsi. Phosphoric acid was added to reach a pH of about 4.7. The mixture was crystallized for 24 hrs in a water bath kept at 30° C., and the resulting complexes were then separated, freeze dried and analyzed.

### Results

The release of genistin from the complexes was tested by HPLC. While in phosphate buffer at pH of 7.2 genistin was not detected in the buffer even after 24 h, the addition of pancreatic amylase resulted in a release of genistin to the media. The results suggested that complexed Isoflavones can be released only upon their arrival to the GI.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1-34. (canceled)

35. A plurality of particles comprising non-covalent complexes comprising starch and an active agent, the particles having a uniform size below 50 μm, wherein the starch is other than vitamin B12 coupled starch.

36. The plurality of the particles of claim 35 having a uniform size below 30 μm.

37. The plurality of the particles of claim 35 having a uniform size below 5 μm.

38. The plurality of the particles of claim 35 having a uniform size below 3 μm.

39. The plurality of particles of claim 35, wherein the active agent is a low molecular weight agent selected from the group consisting of poorly water-soluble agents and amphiphilic agents.

40. The plurality of particles of claim 39, wherein the poorly water-soluble or amphiphilic agent is selected from the group consisting of drugs, peptides, fatty acids, phytoestrogens, steroids, anti-inflammatory agents, antibacterial agents, pro-biotic compounds, vitamins, nutrients, and flavors.

41. The plurality of particles of claim 40, wherein the drug is an anti-cancer drug.

42. The plurality of particles of claim 40, wherein the fatty acid is an ω-3 fatty acid.

43. A suspension comprising the plurality of particles according to claim 35.

44. A dry composition comprising the plurality of particles of claim 35.

45. The dry composition of claim 44, wherein the particles have a uniform size that is below 30 μm.

46. The dry composition of claim 44 formulated in a form selected from the group consisting of tablets, capsules, powders, and pellets.

47. A method for preparing a plurality of particles comprising non-covalent complexes comprising starch and an active agent, which method comprises:
dissolving starch in a first solution having a basic pH to yield a starch solution;
dissolving an active agent in a second solution having a basic pH to yield an active agent solution;
mixing the starch and active agent solutions together to yield a mixture of the starch and the active agent;
feeding the mixture through a first opening into a high-pressure dual feed homogenizer;
feeding an acidic solution having an acidic pH through a second opening into the high-pressure dual feed homogenizer, wherein the feeding of the acidic solution is adjusted to produce a suspension having a pH in the range of about 4 to about 5 and comprising a plurality of particles comprising non-covalent complexes comprising the starch and active agent, with the particles having a uniform size that is below 50 μm; and
optionally, drying the suspension to obtain dry particles.

48. The method of claim 47, wherein the active agent is a low molecular weight agent selected from the group consisting of poorly water-soluble agents and amphiphilic agents.

49. The method of claim 48, wherein the poorly water-soluble or amphiphilic agent is selected from the group consisting of drugs, peptides, fatty acids, phytoestrogens, steroids, pro-biotic compounds, anti-inflammatory agents, antibacterial agents, vitamins, nutrients, and flavors.

50. The method of claim 47, wherein the starch is dissolved in the first solution at a temperature of about 85° C. to 95° C.

51. A method for treating cancer in a subject comprising administering to the subject in need thereof a therapeutically effective amount of the dry composition of claim 44.

52. The method of claim 51, wherein administering the dry composition is performed by oral administration.

53. A method for providing a fatty acid to a subject comprising administering to the subject in need thereof a therapeutically effective amount of the dry composition of claim 44.

54. The method of claim 53, wherein the dry composition is administered orally.
